SEQUENCE LISTING (6 sequences)

SEQ ID NO: 1 - DNA, 24 bp, Artificial sequence
  Synthetic oligonucleotide
aacataaagtctgcaacatggaag

SEQ ID NO: 2 - DNA, 20 bp, Artificial sequence
  Synthetic oligonucleotide
tttgatgggtgaggaatggg

SEQ ID NO: 3 - DNA, 22 bp, Artificial sequence
  Synthetic oligonucleotide
cagagctattggaatcacaggg

SEQ ID NO: 4 - DNA, 21 bp, Artificial sequence
  Synthetic oligonucleotide
tcgctcatacttcactgttgg

SEQ ID NO: 5 - DNA, 21 bp, Artificial sequence
  Synthetic oligonucleotide
gaggctgacttgaccgataag

SEQ ID NO: 6 - DNA, 21 bp, Artificial sequence
  Synthetic oligonucleotide
tgtctcattcagaagggcaac

(19) United States
(12) Patent Application Publication
(10) Pub. No.: US 2013/0267594 A1
(43) Pub. Date: Oct. 10, 2013

(54) PROLYL HYDROXYLASE INHIBITORS AS RADIATION MITIGATORS AND RADIATION PROTECTORS

(71) Applicants: Brendan D. Price, Boston, MA (US); Yingli Sun, Somerville, MA (US)

(72) Inventors: Brendan D. Price, Boston, MA (US); Yingli Sun, Somerville, MA (US)

(73) Assignee: DANA-FARBER CANCER INSTITUTE INC., BOSTON, MA (US)

(21) Appl. No.: 13/820,872

(22) PCT Filed: Sep. 14, 2011
(86) PCT No.: PCT/US11/51541

§ 371 Date: Jun. 27, 2013

Related U.S. Application Data
(60) Provisional application No. 61/382,710, filed on Sep. 14, 2010.

(51) Int. Cl. A61K 41/00

(52) U.S. Cl. CPC A61K 41/00
USPC 514/547; 435/375

(57) ABSTRACT

A method to inhibit tissue injury in a subject resulting from exposure to radiation is provided. The method involves administering to the subject a prolyl hydroxylase (PH) inhibitor in an amount effective to inhibit tissue injury caused by radiation exposure of the subject. The invention also involves a screen for PH inhibitors that inhibit tissue damage resulting from exposure to radiation. Compounds that inhibit the enzymatic activity of PHs are tested for their ability to protect against radiation damage.

### RELATED APPLICATIONS

This application claims the benefit under 35 U.S.C. §119(e) of U.S. provisional application Ser. No. 61/382,710, filed Sep. 14, 2010, the content of which is incorporated by reference herein in its entirety.

### FEDERALLY SPONSORED RESEARCH

This invention was made with Government support under NCI/NIH Grant CA64585 and NIAID/NIH Grant U19AI067751. Accordingly, the Government has certain rights in this invention.

### BACKGROUND OF THE INVENTION

Exposure of humans to radiation can cause serious harm, even death. Exposure can be accidental, resulting, for example, from a radiation leak at a nuclear power plant. Exposure also can be intentional, resulting, for example, from an act of tenor. The most common circumstance of radiation exposure results from medical interventions, such as for the treatment of cancer. Radiation in this context can be local or systemic. When applied locally, radiation can nonetheless cause unwanted injury to healthy tissue in the pathway of the radiation. When applied systemically (i.e., total body irradiation), low doses can lead to bone marrow damage and gastrointestinal tract toxicity. High doses of total body irradiation can lead to non-recoverable bone marrow damage, gut and lung toxicity, and sometimes death. There is a need for effective treatments which protect healthy tissues and inhibit the acute and chronic effects of exposure to ionizing radiation.

### SUMMARY OF THE INVENTION

It has been discovered, surprisingly, that prolyl hydroxylase (PH) inhibitors can protect cells from the cytotoxic effects of ionizing radiation exposure, even when administered post-exposure. The invention involves the use of PH inhibitors to inhibit injury to healthy tissue resulting from exposure to radiation, whether arising from accidental or intentional exposure. The invention permits acute treatment of those exposed unexpectedly to tissue damaging amounts of radiation. The invention permits conventional doses of radiation therapy in medical treatments, with less damage to healthy tissue. The invention also permits an increase to conventional doses of radiation therapy in medical treatments, without an increase in damage to healthy tissue. These and other aspects of the invention are described below.

According to one aspect of the invention, a method to inhibit tissue injury in a subject during, before or after exposure to radiation is provided. The method involves administering to a subject in need thereof a prolyl hydroxylase (PH) inhibitor in an amount effective to inhibit tissue injury in the subject caused by radiation exposure. In important embodiments, the tissue is non-cancerous. The radiation exposure may comprise accidental or deliberate radiation exposure. The radiation exposure may be radiation therapy, for example, local radiation therapy or total body irradiation. In some embodiments, the inhibitor is administered between 1 day before and 3 days after exposure of the subject to the radiation. In some embodiments, the inhibitor is administered between 12 hours before and 3 days after exposure of the subject to the radiation. In some embodiments, the inhibitor is administered between 12 hours before and two days after exposure of the subject to the radiation. In some embodiments, the inhibitor is administered only after the subject has been exposed to radiation, such as least 1 hour, at least 12 hours, at least 24 hours, or at least 48 hours after radiation exposure, but not more than 3 days after radiation exposure.

PH inhibitors represent a new class of molecules for use in the mitigation of radiation damage. The inhibitor may be a non-specific PH inhibitor or may be a specific PH inhibitor. In some embodiments, the PH inhibitor inhibits a HIF1α-prolyl hydroxylase or a histone lysine demethylase (KDM). In some embodiments, the PH inhibitor upregulates HIF1α. Examples of PH inhibitors include dimethyloxalylglycine (DMOG), N-oxalylglycine (NOG), desferrioxamine (DFO), FG-4383, F-0041, FG-2216, FG-4592, S956711, ethyl-3,4 dihydroxy-benzoate (EDHB), TM6089, TM655, TM6008 and 8-hydroxyquinoline derivatives. The inhibitor may be administered systemically or locally.

According to one aspect of the invention, a method to inhibit the number of double-stranded DNA breaks (DSB) in cells is provided. The method comprises contacting cells with a prolyl hydroxylase (PH) inhibitor in an amount effective to inhibit the number of double-stranded DNA breaks (DSB) in the cells, wherein the cells are or will be exposed to radiation causing double stranded DNA breaks. In important embodiments, the cells are is non-cancerous. By inhibiting the number of double stranded breaks, it is meant that less double stranded breaks are detectable following the same conditions of radiation exposure in cells treated with the prolyl hydroxylase (PH) inhibitor than in cells not treated with the prolyl hydroxylase (PH) inhibitor. Without wishing to be bound by any theory, it is believed that the decrease in detectable double stranded breaks is likely due to an improvement in the rate or extent of repair of double stranded breaks caused by the radiation.

The radiation causing double-stranded DNA breaks may comprise accidental or deliberate radiation exposure. In some embodiments, the cells are contacted with the PH inhibitor between 1 day before and three days after exposure of the cells to the radiation. In some embodiments, the cells are contacted with the PH inhibitor between 12 hours before and 3 days after exposure of the cells to the radiation. In some embodiments, the cells are contacted with the PH inhibitor between 12 hours before and two days after exposure of the cells to the radiation. In some embodiments, the cells are contacted with the PH inhibitor only after the cells have been exposed to radiation, such as least 1 hour, at least 12 hours, at least 24 hours, or at least 48 hours after radiation exposure, but not more than 3 days after radiation exposure.

In some embodiments, the PH inhibitor reduces the number of DSB in the cells by at least 5%, 10%, 20%, 50%, 75% or 100% as compared to the number of DSB in control cells not exposed to conditions causing DSB. The inhibitor may be a non-specific PH inhibitor. In some embodiments, the prolyl hydroxylase is a HIF1α-prolyl hydroxylase or a histone lysine demethylase (KDM). In some embodiments, the PH inhibitor upregulates HIF1α. The inhibitor may be selected from the group consisting of: dimethyloxalylglycine (DMOG), N-oxalylglycine (NOG), desferrioxamine (DFO), FG-4383, F-0041, FG-2216, FG-4592, S956711, ethyl-3,4 dihydroxy-benzoate (EDHB), TM6089, TM655, TM6008 and 8-hydroxyquinoline derivatives.

The subject is an animal, typically a mammal. In one aspect, the subject is a dog, a cat, a horse, a sheep, a goat, a cow or a rodent. In important embodiments, the subject is a human. The cells may be derived from any of the foregoing animals. The cells may be contacted with the PH inhibitor either in vitro or in vivo.

Each of the embodiments and aspects of the invention can be practised independently or combined. Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

These and other aspects of the inventions, as well as various advantages and utilities will be apparent with reference to the Detailed Description. Each aspect of the invention can encompass various embodiments as will be understood. All documents identified in this application are incorporated in their entirety herein by reference.

### BRIEF DESCRIPTION OF THE DRAWINGS

FIG. 1 shows that demethylation of lysine 9 on histone 3 by lysine demethylase 4D (KDM4D) sensitizes cells to irradiation (IR).

FIG. 2 shows that DMOG inhibits the KDM4D histone demethylase. HeLa cells were transiently transfected with either vector or KDM4D, followed by incubation with either solvent (PBS, control) or DMOG (100 μM) and then visualized by fluorescent microscopy.

FIG. 3 shows that DMOG increases radioresistance. Clonogenic cells survival assays were performed on MEF cells that were incubated with DMOG (100 mM; circles) or solvent (PBS: squares) and then irradiated.

FIG. 4 shows that DMOG protects Suv39h1/2−/− cells. FIG. 4A shows a Western blot of MEFs incubated with or without DMOG (100 μM/2hours). FIG. 4B shows the impact of DMOG on the survival of Suv39h1/2+/+ and Suv39h1/2−/− cells.

FIG. 5 shows that DMOG is a radiation mitigator. MEFs were incubated with DMOG for 1-24 hours, 24-48 hours or 48-72 hours post-irradiation, and clonogenic cells survival assays were performed.

FIG. 6 shows that DMOG improves the survival of C57BL/6J (FIG. 6A) and Balb/c (FIG. 6B) mice after TBI. Mice (10 per group) were irradiated (8 Gy TBI) and survival monitored over 30 days. DMOG (100 mg/kg) or saline was administered ip 4 hrs before TBI, and 12 hrs and 36 hrs after TBI.

FIG. 7 shows that upregulation of Hif1α promotes radioprotection. In FIG. 7A MCF-7 cells were incubated in DMOG (1 mM) or CoCl₂ (200 μM) for 10 hr or exposed to 10 Gy of ionizing radiation (IR) for the indicated times. Cell extracts were prepared and examined by western blot (WB) analysis to monitor levels of Hif1α. β-actin levels are shown to demonstrate equal loading. In FIG. 7B, MCF-7 cells were incubated in DMOG (1 mM) or CoCl₂ (200 μM) for the indicated times. Cells were irradiated (10 Gy) at time zero where indicated. Cell extracts were prepared and examined by western blot (WB) analysis to monitor levels of Hif1α. β-actin levels are shown to demonstrate equal loading. In FIG. 7C, MEFs were preincubated in DMOG (1 mM) for 1 hr, followed by exposure to IR as indicated. 24 hours later, DMOG was removed and clonogenic cell survival assays carried out. =DMOG, ∘=solvent (PBS).

FIG. 8 shows that HIF1α increases transcription of the CHD4 and MTA3 genes. In FIG. 8A MCF-7 cells stably expressing either a non-specific shRNA (shRNA^{Con}) or shRNA targeting Hif1α (shRNA^{Hif1α}) were exposed to DMOG (1 mM) or CoCl₂ (200 μM) for 24 hrs. Cell extracts were prepared and then examined by western blot (WB) analysis to monitor levels of Hif1α. β-actin levels are shown to demonstrate equal loading. In FIGS. 8B-8E MCF-7 cells stably expressing either a non-specific shRNA (shRNA^{Con}) or shRNA targeting Hif1α (shRNA^{Hif1α}) were exposed to solvent (open bars) or CoCl₂ (200 μM; filled bars) for 8 hr. mRNA levels for the indicated genes were measured by RT-PCR as described in methods. Results+SE (n=3). In FIG. 8F MCF7 cells stably expressing either a non-specific shRNA (shRNA^{Con}) or shRNA targeting Hif1α (shRNA^{Hif1α}) were exposed to CoCl₂ (200 μM) for indicated number of hours. Cell extracts were prepared and then examined by western blot (WB) analysis to monitor levels of CHD4, MTA3 and Hif1α. β-actin levels are shown to demonstrate equal loading.

FIG. 9 shows the Radiosensitivity of cells lacking Hif1α or CHD4. In FIG. 9A MCF-7 cells stably transfected with control (shCon) or Hif1α (shHif1α) shRNA or in FIG. 9B HEK293T cells stably transfected with control (shCon) or CHD4 (shCHD4) shRNA were irradiated as indicated and clonogenic cell survival assays carried out. Western blot analysis of CHD4 protein levels shown in FIG. 9B (inset). Results+SE (n=3).

FIG. 10 shows that DMOG inhibits lysine demethylases and increases levels of the Suv39h1 methyltransferase. In FIG. 10A MEFs were incubated in either solvent (Control: DMSO) or DMOG (1 mM) for 24 hr. H3K9me3 levels were detected by immunofluorescent staining with antibody to H3K9me3 and co-stained with DAPI to locate nuclear DNA. In FIG. 10B HEK293T cells were transiently transfected with vector or KDM4A and allowed to recover for 30 hrs. Cells were either untreated or incubated with DMOG (1 mM) for 24 hrs. H3K9me3 levels were detected by immunofluorescent staining with antibody to H3K9me3. In FIG. 10C MCF-7 cells were incubated in increasing concentrations of DMOG or CoCl₂ (200 μM) for 24 hrs. Cell extracts were prepared and examined by western blot (WB) analysis to monitor levels of Hif1α and Suv39h1. β-actin levels are shown to demonstrate equal loading. In FIG. 10D MCF-7 cells were incubated in DMOG (1 mM) or CoCl₂ (200 μM) for the indicated time. Cell extracts were prepared and examined by western blot (WB) analysis to monitor levels of Hif1α and Suv39h1. β-actin levels are shown to demonstrate equal loading. In FIG. 10E MCF-7 cells expressing either a non-specific shRNA (shRNA^{Con}) or shRNA targeting Hif1α (shRNA^{Hif1α}) were incubated with DMOG or CoCl₂ (200 μM) for 24 hrs. Cell extracts were prepared and examined by western blot (WB) analysis to monitor levels of Hif1α and Suv39h1. β-actin levels are shown to demonstrate equal loading.

FIG. 11 shows that DMOG protects cells from radiation by stabilizing the Hif1α protein. In FIG. 11A MEFs expressing a non-specific shRNA (∘, ; sh^{Con}) or shRNA targeting Hif1α (ε, ▪; sh^{Hif1α}) were untreated (∘, □) or preincubated for 1 hr in DMOG (, ▪; 1 mM), irradiated at the indicated dose and allowed to recover for 24 hr. DMOG was removed by medium exchange and clonogenic cell survival assays carried out following 10-12 days growth in culture. Results+SE (n=6). In FIG. 11B wild type MEFs or MEFs derived from mice with a double knockout of Suv39h1 and Suv39h2 (MEF-Suv^{DKO}) were incubated with DMOG (1 mM) or CoCl₂ (200 μM) for 24 hrs. Cell extracts were prepared and examined by western blot (WB) analysis to monitor levels of Hif1α. β-actin levels are shown to demonstrate equal loading. In FIG. 11C MEFs derived from mice with a double knockout of Suv39h1 and Suv39h2 (MEF-Suv^{DKO}) were incubated with solvent (∘) or DMOG (; 1 mM) for 1 hr, irradiated at the indicated dose and allowed to recover for 24 hrs. DMOG was removed by medium exchange, cells allowed to recover for 10-12 days, and clonogenic cell survival assays carried out. Results+SE (n=3).

### DETAILED DESCRIPTION OF THE INVENTION

The present invention, in one aspect, relates to the surprising discovery that prolyl hydroxylase (PH) inhibitors can protect cells from the cytotoxic effects of ionizing radiation exposure. PH inhibitors represent a new class of agents that can be used to protect individuals from unwanted tissue injury resulting from exposure to radiation.

The PH inhibitors are used to treat subjects in need of protection against unwanted tissue damage caused by exposure to radiation. As used herein, a subject in need of PH inhibitor therapy, therefore, is any subject exposed to levels of radiation capable of causing unwanted tissue injury. In some embodiments, the subject has already has been exposed to the radiation prior to the PH inhibitor therapy. In some embodiments, the subject is in need of prophylactic treatment with PH inhibitors in anticipation of future radiation exposure. In some embodiments, the subject is being exposed to radiation during PH inhibitor therapy. Radiation exposure includes, but is not limited to, accidental exposure, exposure resulting from a nuclear attack, and medical radiation therapy such as local therapy and low and high dose total body irradiation. In some embodiments, the subject has cancer, and has undergone, is undergoing or will undergo radiation therapy.

Exposure to radiation is toxic at low doses and life threatening at high doses. The tissues which are most vulnerable to radiation-induced damage include the hematopoietic system and the gastrointestinal tract (GI). Moderate doses of radiation can cause a rapid reduction in blood cells counts, including loss of circulating lymphocytes and a reduction in mitotically active hematopoietic progenitor cells. Reduction in blood cell count is associated, among other things, with increased risk of infections and cancer development. Higher doses of radiation can lead to more severe and often non-recoverable bone marrow damage, resulting from loss of bone marrow stem cell populations. At lower doses of ionizing radiation, GI effects can be relatively mild. At higher doses (above 5 Gy), however, GI effects can include loss of villi structure, death of GI stem cells and breakdown of the GI tract, leading to bacterial infection, internal bleeding and in some instances irreversible breakdown of GI tract integrity.

Accordingly, the invention involves inhibiting radiation-induced tissue injury of any type. In important embodiments, the tissue is non-cancerous tissue. The tissue injury may be, for example, any or all of soft tissue radionecrosis, osteoradionecrosis, radiation mucositis, dermatitis, enteritis, laryngeal radionecrosis, and/or cystitis. In some embodiments, radiation-induced tissue injury is a decrease in blood cell count, loss of bone marrow stem cell populations, a decrease in villi structure of the GI tract, loss of GI stem cells, an increase in the number of double-stranded DNA breaks (DSB) in the tissue and/or a decrease in the extent of DNA repair in the tissue.

The compounds useful in the methods of the invention are PH inhibitors. Prolyl hydroxylases (PHs) are members of an extended family of Fe(II) and 2-oxoglutarate dependent dioxygenases. Dioxygenases share a common catalytic mechanism and can hydroxylate proteins on multiple amino-acids, including proline, lysine, asparagine, aspartic acid and tryptophan residues. There are 9 dioxygenase sub-families, of which 2 play critical roles in the cells response to ionizing radiation. These are Histone Lysine Demethylases (KDMs) and HIF prolyly hydroxylase (HIF-PH) families. PHs require oxoglutarate, iron, and ascorbic acid as cofactors and this property renders them susceptible to pharmacological attack. In some embodiments, the PH inhibitor inhibits more than one family of PHs, and is therefore a non-specific inhibitor. Examples of PH inhibitors, include, but are not limited to dimethyloxalylglycine (DMOG), N-oxalylglycine (NOG), desferrioxamine (DFO), FG-4383, F-0041, FG-2216, FG-4592, S956711, ethyl-3,4 dihydroxy-benzoate (EDHB), TM6089, TM655, TM6008 and 8-hydroxyquinoline derivatives.

Without being bound by theory, it has now been found that inhibition of PHs, in particular the HIF1α-prolyl hydroxylase (HIF-PH) sub-family and the lysine demethylase sub-family (KDM) subfamily, will activate cell based signaling systems which function to mitigate the cytotoxic effects of ionizing radiation exposure. Inhibition of the HIF-PH will block HIF1α degradation, leading to the transcriptional activation of genes which promote adaptation to hypoxia, increase erythropoietin production, improve survival of endothelial and mesenchymal stem/progenitor cells, reduce inflammatory cytokines and protection the vasculature from radiation damage. Inhibition of KDM will increase levels of histone 3 lysine 9 methylation (H3K9me3) across the chromatin, promoting both increased activation of the ATM kinase and changes in chromatin structure which can facilitate repair of DSB which are in compacted, inaccessible regions of the chromatin. Thus, PH inhibitors can protect tissues and have a significant impact on improving the survival of subjects exposed to radiation.

The PH inhibitors are administered to inhibit radiation-induced tissue injury. As used herein, the term “inhibit” refers to a reduction in the extent of radiation-induced tissue damage versus that which would occur absent the PH inhibitor treatment. As such, the reduction in the extent of radiation-induced tissue damage may be evaluated in terms of an improvement in the health of the tissue in treated subjects. An improvement in the health of tissues of treated subjects may be determined by examining the health of the tissue in treated subjects versus the health of tissue in control subjects (i.e., subjects receiving the same amount of radiation exposure treated subjects but not receiving the PH inhibitor therapy). The health of the tissue may be measured by any variety of methods known to those of ordinary skill in the art, including direct and indirect measurements. Direct measurements are those such as measuring cell count. Indirect measurements are those such as measuring symptoms.

Radiation tissue damage can include soft tissue radionecrosis, osteoradionecrosis, radiation mucositis, dermatitis, enteritis, laryngeal radionecrosis, cystitis, etc. It also can include an impaired ability to heal following a wound, including a surgical wound. Radiation injuries can involve cell death. Radiation damage also can lead to a deterioration in small blood vessels. In bone, blood vessels also can be damaged by radiation, accompanied by death of bone cells. These changes can lead to osteonecrosis and porosis. All of these conditions can be observed, and there are well known animal models for determining such radiation tissue damage, including, for example, animal models for soft tissue necrisos, mucositis and scleroderma. In some embodiments, the tissue injury measured may be necrosis of the tissue, a decrease in blood cell count, and/or loss of villi structure of the GI tract. In some embodiments, the tissue injury may be measured by evaluating the function of the GI tract or some other organ affected by the radiation treatment. In some embodiments, the tissue injury may be measured by evaluating the extent of double stranded breaks following exposure to radiation and PH inhibitor treatment.

The PH inhibitors are administered in effective amounts. An effective amount is a dose sufficient to provide a medically desirable result and can be determined by one of skill in the art using routine methods. In the treatment of radiation-induced tissue damage, an effective amount will be that amount necessary to inhibit the tissue damage caused by exposure to radiation. In some embodiments, an effective amount is an amount which results in any improvement in the condition being treated. In some embodiments, an effective amount may depend on the type and extent of radiation exposure and/or use of one or more additional therapeutic agents. However, one of skill in the art can determine appropriate doses and ranges of PH inhibitors to use, for example based on in vitro and/or in vivo testing and/or other knowledge of compound dosages. It should be appreciated that in some embodiments, the PH inhibitors described herein may be administered in dosages that inhibit radiation-caused injury of non-cancerous tissues and cells, without materially interfering with the killing of cancerous tissues and cells.

When administered to a subject, effective amounts of PH inhibitor(s) will depend, of course, on the particular tissue injury being treated; the severity of the injury; individual patient parameters including age, physical condition, size and weight, concurrent treatment, frequency of treatment, and the mode of administration. These factors are well known to those of ordinary skill in the art and can be addressed with no more than routine experimentation. In some embodiments, a maximum dose is used, that is, the highest safe dose according to sound medical judgment.

An effective amount typically will vary from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, from about 10.0 mg/kg to about 150 mg/kg in one or more dose administrations, for one or several days (depending of course of the mode of administration and the factors discussed above).

Actual dosage levels of the PH inhibitor(s) can be varied to obtain an amount that is effective to achieve the desired therapeutic response for a particular patient, compositions, and mode of administration. The selected dosage level depends upon the activity of the particular compound, the route of administration, the severity of the radiation exposure, the tissue being treated, and prior medical history of the patient being treated. However, it is within the skill of the art to start doses of the compound at levels lower than required to achieve the desired therapeutic effort and to gradually increase the dosage until the desired effect is achieved.

The PH inhibitors may be administered before, during or after exposure of the subject to levels of radiation causing tissue damage. In some embodiments, the PH inhibitors are administered before radiation exposure, but close enough in time to the radiation exposure to inhibit radiation-induced tissue damage. In some embodiments, the PH inhibitor is administered any time up to 1 day before the radiation exposure. In some embodiments, the PH inhibitor is administered between 1 and 24 hours before radiation exposure. In some embodiments, the PH inhibitor is administered within 12 hours of radiation exposure. The PH inhibitor may also be administered during radiation exposure. In some embodiments, the PH inhibitor is administered after radiation exposure, yet close enough in time to the radiation exposure to have the desired effect of protecting tissue from radiation-induced tissue injury. In some embodiments, the PH inhibitor may be administered any time up to 3 days post-exposure. In some embodiments, the PH inhibitor is administered between 1-60 hours following radiation exposure. In some embodiment, the PH inhibitor is administered within 24 or 48 hours of radiation exposure. In some embodiments, the subject has cancer and the PH inhibitor is administered at least 1 hour, at least 12 hours, at least 24 hours, or at least 48 hours following radiation therapy, but not more than 72 hours following radiation therapy.

According to some aspects of the invention, a method to reduce the number of double-stranded DNA breaks (DSB) in cells is provided. In some embodiments, the cells are non-cancerous cells. Cells exposed to radiation are prone to acquire double stranded breaks in DNA. DNA double-stranded breaks jeopardize a chromosome's physical integrity essential for its correct segregation during mitosis and meiosis as well as its informational redundancy critical for maintaining accurate encoding of cellular components. The accumulation of these breaks in a cell can eventually trigger apoptosis and cell death. Without being bound by theory, it is hypothesized that PH inhibitors favorably affect the extent and/or the kinetics of DNA repair.

Accordingly, non-cancerous cells that are exposed to, have been exposed to, or will be exposed to radiation are contacted with an effective amount of a PH inhibitor to reduce or inhibit the number of DSB in the cells. The cells, thus, are treated with the PH inhibitor before, during or after radiation exposure. In some embodiments, the inhibitor reduces the number of DSB in the cell by at least 5%, at least 10%, at least 20%, at least 30%, or at least 50% as compared to the number of DSB in control cells. The extent of reduction or inhibition in the number of DSB in the cells can be measured using assays well-known in the art, including but not limited to comet assays and pulsed-field gel electrophoresis. The control cells are typically identical to the treated cells in all parameters, except the control cells are not exposed to the radiation.

The PH inhibitors and pharmaceutical compositions containing PH inhibitors are administered to a subject by any suitable route. For example, the compositions can be administered orally, including sublingually, rectally, parenterally, intracisternally, intravaginally, intraperitoneally, topically and transdermally (as by powders, ointments, or drops), bucally, or nasally. The term “parenteral” administration as used herein refers to modes of administration other than through the gastrointestinal tract, which include intravenous, intramuscular, intraperitoneal, intrasternal, intramammary, intraocular, retrobulbar, intrapulmonary, intrathecal, subcutaneous and intraarticular injection and infusion. Surgical implantation also is contemplated, including, for example, embedding a composition of the invention in the body such as, for example, in the brain. In some embodiments, the compositions may be administered systemically. In some embodiments, the PH inhibitors may be contacted with or administered directly to non-cancerous cells.

Pharmaceutical compositions of the invention for parenteral injection comprise pharmaceutically acceptable sterile aqueous or nonaqueous solutions, dispersions, suspensions, or emulsions, as well as sterile powders for reconstitution into sterile injectable solutions or dispersions just prior to use. Examples of suitable aqueous and nonaqueous carriers, diluents, solvents, or vehicles include water ethanol, polyols (such as, glycerol, propylene glycol, polyethylene glycol, and the like), and suitable mixtures thereof, vegetable oils (such, as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions also can contain preservatives, wetting agents, emulsifying agents, and dispersing agents. Prevention of the action of microorganisms can be ensured by the inclusion of various antibacterial and antifungal agents, for example, paraben, chlorobutanol, phenol sorbic acid, and the like. It also may be desirable to include isotonic agents such as sugars, sodium chloride, and the like. Prolonged absorption of the injectable pharmaceutical form can be brought about by the inclusion of agents which delay absorption, such as aluminum monostearate and gelatin.

In some cases, in order to prolong the effect of the drug, it is desirable to slow the absorption of the drug from a subcutaneous or intramuscular injection. This result can be accomplished by the use of a liquid suspension of amorphous materials with poor water solubility. Delayed absorption of a parenterally administered drug also is accomplished by dissolving or suspending the drug in an oil vehicle Likewise, injectable depot forms are made by forming microencapsule matrices of the drug in biodegradable polymers such a polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations also are prepared by entrapping the drug in liposomes or microemulsions which are compatible with body tissue.

The invention provides methods for oral administration of a pharmaceutical composition of the invention. Oral solid dosage forms are described generally in Remington's Pharmaceutical Sciences, 18th Ed., 1990 (Mack Publishing Co. Easton Pa. 18042) at Chapter 89. Solid dosage forms for oral administration include capsules, tablets, pills, powders, troches or lozenges, cachets, pellets, and granules. Also, liposomal or proteinoid encapsulation can be used to formulate the present compositions (as, for example, proteinoid microspheres reported in U.S. Pat. No. 4,925,673). Liposomal encapsulation may include liposomes that are derivatized with various polymers (e.g., U.S. Pat. No. 5,013,556).

In such solid dosage forms, the active compound is mixed with, or chemically modified to include, at least one inert, pharmaceutically acceptable excipient or carrier. The excipient or carrier may permit increased uptake of the compound, overall stability of the compound and/or circulation time of the compound in the body. Excipients and carriers include, for example, sodium citrate or dicalcium phosphate and/or (a) fillers or extenders such as starches, lactose, sucrose, glucose, cellulose, modified dextrans, mannitol, and silicic acid, as well as inorganic salts such as calcium triphosphate, magnesium carbonate and sodium chloride, and commercially available diluents such as FAST-FLO®, EMDEX®, STA-RX 1500®, EMCOMPRESS® and AVICEL®, (b) binders such as, for example, methylcellulose ethylcellulose, hydroxypropyhnethyl cellulose, carboxymethylcellulose, gums (e.g., alginates, acacia), gelatin, polyvinylpyrrolidone, and sucrose, (c) humectants, such as glycerol, (d) disintegrating agents, such as agar-agar, calcium carbonate, potato or tapioca starch, alginic acid, certain silicates, sodium carbonate, starch including the commercial disintegrant based on starch, EXPLOTAB®, sodium starch glycolate, AMBERLITE®, sodium carboxymethylcellulose, ultramylopectin, gelatin, orange peel, carboxymethyl cellulose, natural sponge, bentonite, insoluble cationic exchange resins, and powdered gums such as agar, karaya or tragacanth; (e) solution retarding agents such a paraffin, (f) absorption accelerators, such as quaternary ammonium compounds and fatty acids including oleic acid, linoleic acid, and linolenic acid (g) wetting agents, such as, for example, cetyl alcohol and glycerol monosterate, anionic detergent surfactants including sodium lauryl sulfate, dioctyl sodium sulfosuccinate, and dioctyl sodium sulfonate, cationic detergents, such as benzalkonium chloride or benzethonium chloride, nonionic detergents including lauromacrogol 400, polyoxyl 40 stearate, polyoxyethylene hydrogenated castor oil 10, 50 and 60, glycerol monostearate, polysorbate 40, 60, 65, and 80, sucrose fatty acid ester, methyl cellulose and carboxymethyl cellulose; (h) absorbents, such as kaolin and bentonite clay, (i) lubricants, such as talc, calcium sterate, magnesium stearate, solid polyethylene glycols, sodium lauryl sulfate, polytetrafluoroethylene (PTFE), liquid paraffin, vegetable oils, waxes, CARBOWAX® 4000, CARBOWAX® 6000, magnesium lauryl sulfate, and mixtures thereof; (j) glidants that improve the flow properties of the drug during formulation and aid rearrangement during compression that include starch, talc, pyrogenic silica, and hydrated silicoaluminate. In the case of capsules, tablets, and pills, the dosage form also can comprise buffering agents.

The solid dosage forms of tablets, dragees, capsules, pills, and granules can be prepared with coatings and shells, such as enteric coatings and other coatings well known in the pharmaceutical formulating art. Liquid dosage forms for oral administration include pharmaceutically acceptable emulsions, solutions, suspensions, syrups, and elixirs. In addition to the active compounds, the liquid dosage forms can contain inert diluents commonly used in the art, such as, for example, water or other solvents, solubilizing agents and emulsifiers, such as ethyl alcohol, isopropyl alcohol ethyl carbonate ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols, fatty acid esters of sorbitan, and mixtures thereof.

Also contemplated herein is pulmonary delivery of the compounds of the invention. The compound is delivered to the lungs of a mammal while inhaling. Contemplated for use in the practice of this invention are a wide range of mechanical devices designed for pulmonary delivery of therapeutic products, including, but not limited to, nebulizers, metered dose inhalers, and powder inhalers, all of which are familiar to those skilled in the art. All such devices require the use of formulations suitable for the dispensing of a compound of the invention. Typically, each formulation is specific to the type of device employed and can involve the use of an appropriate propellant material, in addition to diluents, adjuvants, and/or carriers useful in therapy.

The present invention is further illustrated by the following Example, which in no way should be construed as further limiting. The entire contents of all of the references (including literature references, issued patents, published patent applications, and co-pending patent applications) cited throughout this application are hereby expressly incorporated by reference.

### EXAMPLE

Cell Culture: HEK293T, MCF-7 (ATCC, NJ) and MEF [42] cells were cultured as previously described. Cells were irradiated using a Cs¹³⁷ irradiator and clonogenic cell survival monitored as previously described. Dimethyloxalylglycine was purchased from Frontier Scientific (Logan, Utah). HEK293T, MCF7, and MEF cells were transfected using Lipofectamine 2000 according to the manufacturer's instructions (Invitrogen, CA), and selected using puromycin. Lentiviral HIF1α shRNA and GFP control shRNA were obtained from The RNAi Consortium (Broad Institute, Cambridge, Mass.). Plasmids were packaged with VSV-G expressing constructs phCMV-G and PCMVδ8.2 plasmid and transfected into HEK293T cells using Lipofectamine 2000. After 48 h, viral particles were harvested from the culture medium and used to prepare stable cell lines expressing GFP or HIF1α shRNA after selection with puromycin. The histone demethylase KDM4A was transiently expressed in HEK293T cells using Fugene-6 (Roche, Ind.) for 30 hr prior to use for experimental protocols.

Cell lysates and Western blot analysis: Antibodies used: mHif1α (Santa Cruz Biotechnology, CA); hHIF1α (Abcam, CA); H3K9me3 (Millipore, N.Y.); β-actin (Cell Signaling Technology, Mass.); CHD4 and MTA3 (Bethyl Laboratories, Tex.); Suv39h1 (Novus, CA). To prepare nuclear lysates, cells were resuspended in 500 μl buffer A (10 mM Tris, pH8.0/1 mM EDTA, pH8.0/150 mM NaCl/0.5% NP-40/1 mM PMSF/5 μg/ml leupeptin/20 μg/ml aprotinin), incubated on ice for 15 min and centrifuged at 500 g for 5 min at 4° C. Supernatant was removed and the pellet resuspended in buffer B (20 mM Hepes, pH7.9/400 mM NaCl/1 mM EDTA/0.5 mM DTT/1 mM PMSF/5 μg/ml leupeptin/20 μg/ml aprotinin), incubated on ice for 15 min and cleared by centrifugation (21,000 g for 5 min at 4° C.). For western blots, equal amounts of protein (Bradford Protein Assay kit, Bio-Rad Laboratories, CA) were separated by electrophoresis and transferred to nitrocellulose membranes (Bio-Rad Laboratories, CA). Membranes were blocked with Odyssey Blocking Buffer (Li-Cor, NB), incubated with primary antibodies for 3 h, and washed in PBST (PBS and 0.1% Tween-20) followed by goat anti-mouse IR Dye-800CW or goat anti-rabbit IR Dye-680CW secondary antibodies (Li-Cor, NB). Imaging was performed using the Li-Cor Odyssey Near Infra Red System, and analyzed using the Odyssey software system 3.0 package (Li-Cor, NB).

RT-PCR: Total RNA was prepared using RNeasy kit (Qiagen, CA). 1 μg RNA and supplied random primers were used to generate cDNA with aid of QuantiTect reverse transcription kit (Qiagen, CA). RT-qPCR was performed using following primers: for HIF1α 5′-AACATAAAGTCTGCAACATGGAAG (SEQ ID NO: 1) and 5′-TTTGATGGGTGAGGAATGGG (SEQ ID NO: 2); for CHD4 5′-CAGAGCTATTGGAATCACAGGG (SEQ ID NO: 3)and 5′-TCGCTCATACTTCACTGTTGG (SEQ ID NO: 4); for MTA3 5′-GAGGCTGACTTGACCGATAAG (SEQ ID NO: 5) and 5′-TGTCTCATTCAGAAGGGCAAC (SEQ ID NO: 6); VEGF primers were used as positive control and 18S RNA primers were used as internal control.

Immunofluorescence: Cells (on cover slides) were fixed in phosphate-buffered saline (PBS) containing 2% paraformaldehyde. Cells were permeabilized in 0.2% Triton X-100 in PBS for 5mins, and then blocked in fetal bovine serum for 20 min, Primary antibodies were prepared in 10% fetal bovine serum supplemented with 0.2% saponin. After a 1 h incubation with primary antibody, cells were washed three times with 0.2% Tween-20 and incubated for 1 h in secondary antibody (conjugated to either Texas Red or FITC; Santa Cruz Biotechnology, CA). Slides were mounted with Fluoromount-G (Southern Biotech, AL). Images were collected with an AxioImager Z1 microscope (Carl Zeiss, Inc.) equipped with a color digital camera (Axiocam MRc Rev.3; Carl Zeiss, Inc.) and Plan Apochromat oil M27 lens (63×, NA 1.4). Acquisition software and image processing used the AxioVision software package (Carl Zeiss, Inc.).

Mice: This study was carried out in strict accordance with the recommendations in the Guide for the Care and Use of Laboratory Animals of the National Institutes of Health. The protocol was reviewed and approved by the Dana-Farber Cancer Institute's Animal Care and Use Committee (Protocol number 06-029). All efforts were made to minimize suffering. Male Balb/c mice were purchased from Taconic (NY, USA) and C57BL/6J mice were purchased from The Jackson Laboratory (ME, USA). For total body irradiation (TBI), mice were irradiated at the dose rate of 110 cGy/min using a ¹³⁷Cs source (Gammacell® 40Exactor, Best Theratronics, Ottawa, Canada). DMOG (dissolved in saline) or saline was administered IP before and after TBI. The mice were given normal water that had undergone reverse osmosis and powdered food (Purina Pico Lab Chow) after TBI. The animals were closely inspected after TBI for ill-health and the moribund animals were sacrificed according to the guidelines in our approved ACUC protocol. The Kaplan-Meier survival curves were plotted for saline-treated and DMOG-treated cohorts. The statistical analysis of the Kaplan-Meier survival curves was performed with Graph Pad Prism Version 5 using Mantel Log-rank test.

### EXAMPLE 1

### Modulating H3K9me3 Levels Modulates Radiosensitivity

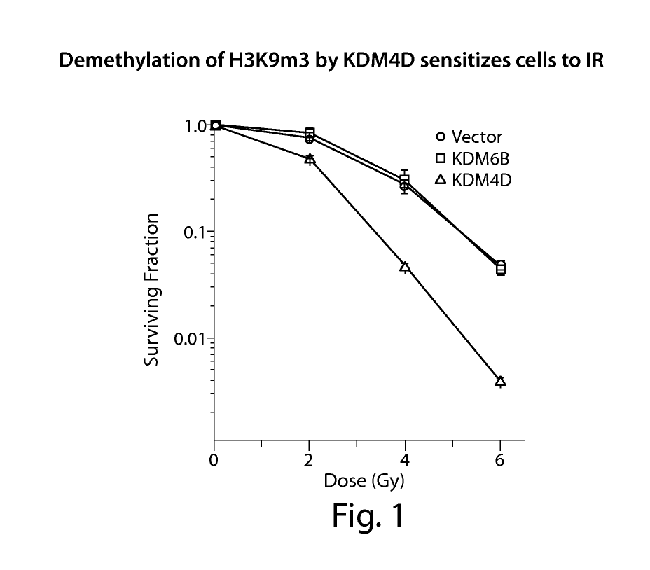

Methylation of histone H3 on lysine 9 (H3K9me3) is required for efficient activation of Tip60, and therefore for the acetylation and activation of ATM. Thus, alterations in the levels of H3K9me3 in the cell alter the ability of cells to activate the Tip60-ATM pathway, and therefore alter the cells sensitivity to IR. The inventors confirmed this by expressing 2 histone demethylases, KDM6B (specific for H3K27me3) or KDM4D (specific for H3K9me3) to alter intracellular histone methylation levels. FIG. 1 clearly shows that demethylation of H3K9me3 by KDM4B leads to increased radiosensitivity, whereas reduced H3K27 methylation did not alter intrinsic radiosensitivity.

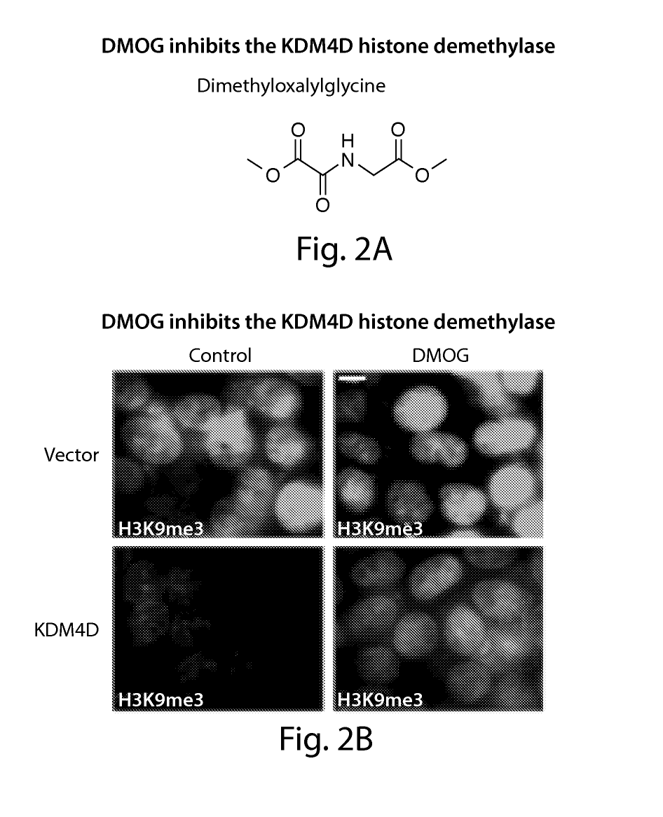

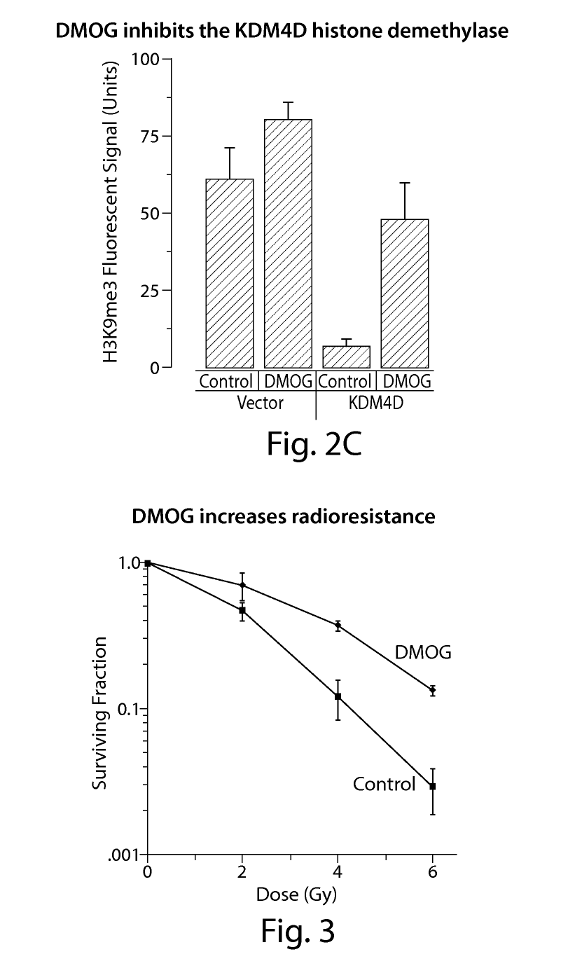

KDMs utilize an Fe-oxo intermediate to form an unstable hydroxy-methyl intermediate, which spontaneously releases formaldehyde, leading to removal of a single methyl group. The ability of 2-oxoglutarate analog, dimethyloxalylglycine (DMOG; FIG. 2A) to inhibit the H3K9me3 demethylase KDM4D was examined. In FIG. 2B, Murine Embryonic Fibroblasts (MEFs) were transfected with empty vector or KDM4D, which specifically demethylates H3K9me3, and the levels of chromatin associated H3K9me3 measured by immunofluorescent staining using an anti-H3K9me3 antibody. In untreated cells, high levels of H3K9me3 were detected, and transient expression of the H3K9me3 demethylase KDM4D essentially demethylated the entire nuclear H3K9me3 signal. Addition of DMOG increased the overall levels of H3K9me3 in cells (FIG. 2B) by approx 31% (FIG. 2C). Importantly, when DMOG was added to cells expressing the KDM4D demethylase, DMOG inhibited the ability of KDM4D to demethylate H3K9me3. Control experiments demonstrated that DMOG did not exhibit significant toxicity over the time course of the experiment, and did not alter the level of expression of the KDM4D enzyme. These results are consistent with the hypothesis that DMOG functions as an inhibitor of histone demethylases. Further, quantification of these results (FIG. 2C), indicates that this assay system has the potential to be exploited for use as a High Content Screening assay to identify specific inhibitors of KDM4D and related demethylases.

FIG. 2C indicated that DMOG on its own could increase the basal levels of H3K9me3 in HeLa cells by 31%. If H3K9me3 is a limiting factor in cells for activation of Tip60 and regulation of DSB repair, then increasing H3K9me3 levels should decrease the sensitivity of the cells to IR. The inventors tested this by incubating MEF cells in DMOG to increase histone methylation levels and then assessing radiation sensitivity using a clonogenic cell survival assay. FIG. 3 demonstrates that MEF cells exposed to DMOG showed increased cell survival after exposure to IR, consistent with DMOG functioning to mitigate the cytotoxic effects of IR in these cells.

Next, the inventors sought confirmation that the effects of DMOG on radiosensitivity were exerted through inhibition of H3K9me3 demethylases rather than through inhibition of other prolyl hydroxylase family members. For example, DMOG inhibits the HIF-PH, which leads to accumulation of HIF1α and transcriptional activation of the hypoxia response. Further, upregulation of the hypoxic response is associated with increased radioresistance. Therefore, the inventors set out to determine if the ability of DMOG to regulate the sensitivity of cells to ionizing radiation was due to its effects on the levels of H3K9 methylation. Suv39h1 and Suv39h2 are the main H3K9me3 methyltransferases in the cell.

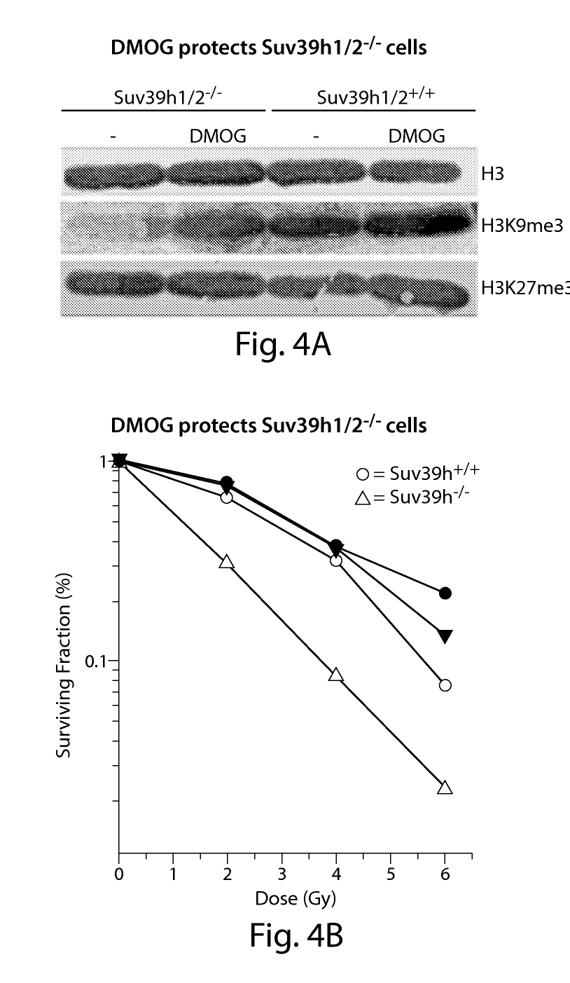

A double knockout of both the Suv39h1 and Suv39h2 methyltransferases in mice leads to reduced levels of H3K9me3 (FIG. 4A) and increased genomic instability. If DMOG functions to inhibit histone demethylase activity, the inventors predicted that it should be more effective in the Suv39h1/2−/− cells compared to the Suv39h1/2+/+ cells.

In FIG. 4A, DMOG increased the levels of H3K9me3 in Suv39h1/2−/− cells to a level similar to those seen in Suv39h1/1+/+ cells. Further, Suv39h1/2−/− cells were more sensitive to IR then Suv39h1/2+/+ cells, (FIG. 4B) consistent with their lower levels of H3K9me3 and reduced activation of ATM and Tip60. When Suv39h1/h2 cells+/+ were treated with DMOG, there was a small, but significant increase in radioresistance. However, the effect was much more dramatic in Suv39h1/h2−/− cells, where DMOG had a much larger radioprotective effects compared to the effects on Suv39h1/h2+/+. Because the 2 cell lines are genetically identical except for the loss of Suv39h1/h2 methyltransferases, and the associated reduction in H3K9me3 levels, the simplest interpretation is that DMOG is regulating radioprotection through inhibition of endogenous demethylases, leading to increased H3K9me3 levels in the cells. However, it is also possible that activation of HIF1α by DMOG inhibition of HIF-PH may also contribute to the protective effects of DMOG on radiation toxicity.

### EXAMPLE 2

### The PH Inhibitor DMOG is a Highly Effective Radiation Mitigator

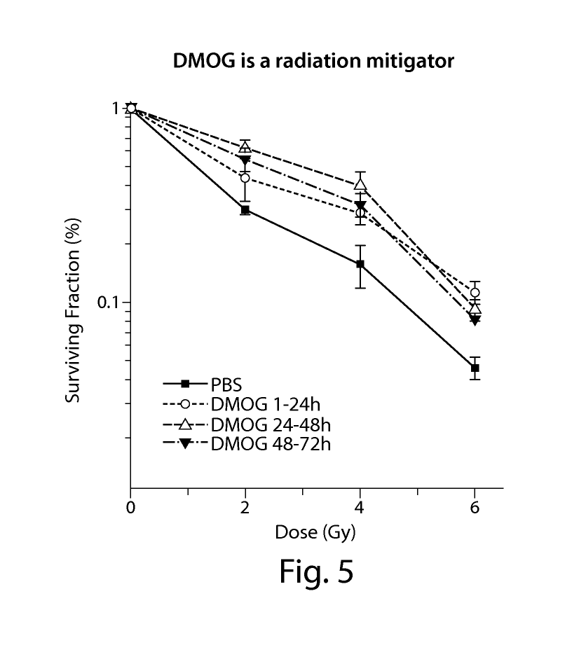

A key requirement for medical countermeasures is that they function as mitigators of radiation toxicity rather than as protective agents. This is critically important for use in case of radiological terrorism events, where therapeutic care can only be proved post-exposure. The inventors therefore tested whether DMOG could function as a radiation mitigator if it was added at least 24 hrs after exposure of cells to ionizing radiation. In FIG. 5, MEFs were irradiated, and the cells incubated in DMOG for either 1-24 hours, 24-48 hours or 48-72 hours following exposure to ionizing radiation. Importantly, DMOG exhibited significant protection even when administered 48 hr after exposure to ionizing radiation. DMOG therefore functions as a radiation mitigator, and demonstrates that PH inhibitors represent a new class of small molecule inhibitors for the rational development of new types of radiation mitigators.

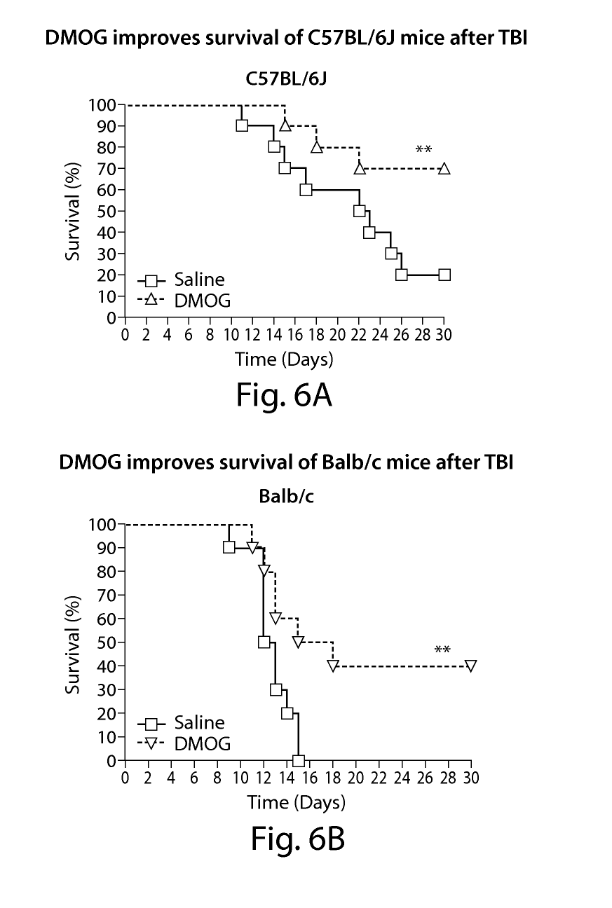

The preliminary observation on the radiation mitigating effects of DMOG in primary fibroblast cell lines prompted the inventors to initiate testing of DMOG in a murine model. The preliminary results are shown in FIG. 6. DMOG was injected ip into either C57BL/6J (FIG. 6A) or Balb/c (FIG. 6B) mice, 2 widely used murine TBI model systems, according to the schedule in FIG. 6. In these experiments, administration of DMOG alone does not cause any toxicity, with 100% of the mice surviving at 30 days (data not shown), in agreement with previous studies using this dose of DMOG. 8 Gy TBI caused 80% and 100% lethality in saline-treated C57BL/6J and Balb/c mice respectively (FIG. 6). DMOG treatment significantly improved the survival of both C57BL/6J and Balb/c mice and rescued them from radiation induced toxicity. FIG. 6 clearly demonstrates that the ability of DMOG to act as a radiation mitigator in cultured cells (FIG. 5) can be replicated in a whole animal model.

### EXAMPLE 3

### Activation of Hif1α by the Prolylhydroxylase Inhibitor Dimethyoxalyglycine Decreases Radiosensitivity

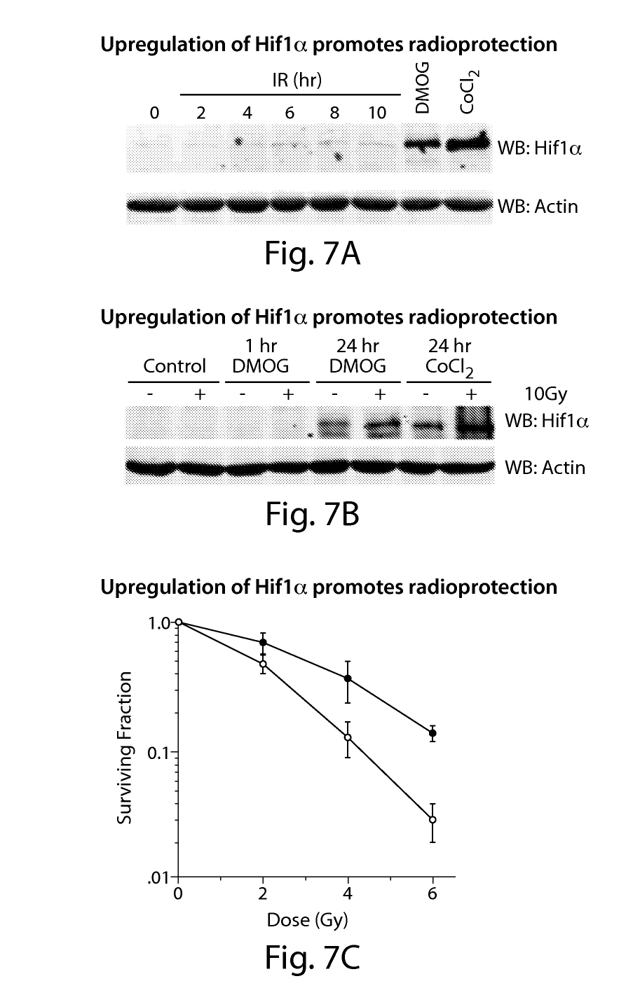

To examine if activation of Hif1α impacted radiosensitivity, the inventors examined if exposure of MCF-7 cells to ionizing radiation (IR) lead to stabilization of the Hif1α transcription factor. MCF-7 cells were exposed to IR or treated with the prolylhydroxylase inhibitors DMOG or CoCl₂. Both DMOG and CoCl₂ caused significant accumulation of Hif1α in MCF-7 cells (FIG. 7A). Exposure to IR alone had only a slight impact on accumulation of Hif1α, indicating that IR does not cause significant stabilization of Hif1α. However, combining IR with either DMOG or CoCl₂ led to much larger accumulation of Hif1α protein than exposure to DMOG or CoCl₂ alone (FIG. 7B). This indicates that DMOG and IR function synergistically to stabilize Hif1α and increase the accumulation of this protein in cells.

Upregulation of Hif1α by treatment with the prolylhydroxylase inhibitor DMOG can improve survival under conditions of stress. Because DMOG functioned synergistically with IR to promote accumulation of Hif1α (FIG. 7B), the inventors determined if the increased levels of Hif1α caused by DMOG affected radiosensitivity. For these experiments, MEFs were used. These were chosen because MEFs, unlike tumor derived or transformed cells, do not express basal levels of Hif1α which can impact intrinsic radiosensitivity. This allows for the direct assessment of the impact of increased Hif1α expression on radiosensitivity. When MEFs were treated for 24 hr with DMOG to increase Hif1α levels, there was a significant decrease in radiosensitivity (FIG. 7C), indicating that DMOG functions as a radioprotector. FIG. 7 therefore indicates that upregulation of Hif1α can protect MEFs from the cytotoxic effects of IR.

Hif1α functions as a transcriptional regulator, suggesting that Hif1α may control the transcription of DNA repair proteins (or apoptotic factors), which can protect cells from radiation damage. Recent work has implicated components of the NuRD deacetylase complex in the cells response to both hypoxia and DSB repair. The NuRD complex, which contains both the CHD4 ATPase and the HDAC2 deacetylase, is required for cells to repair and survive IR-induced DNA damage. Importantly, expression of the MTA1 sub-unit of NuRD is induced under hypoxic conditions, and both MTA1 and histone deacetylases contribute to Hif1α stability. This indicates that components of the NuRD complex may be upregulated by Hif1α. The inventors examined if components of the NuRD complex were increased by hypoxia mimics and if this induction contributed to the observed radioprotection by DMOG.

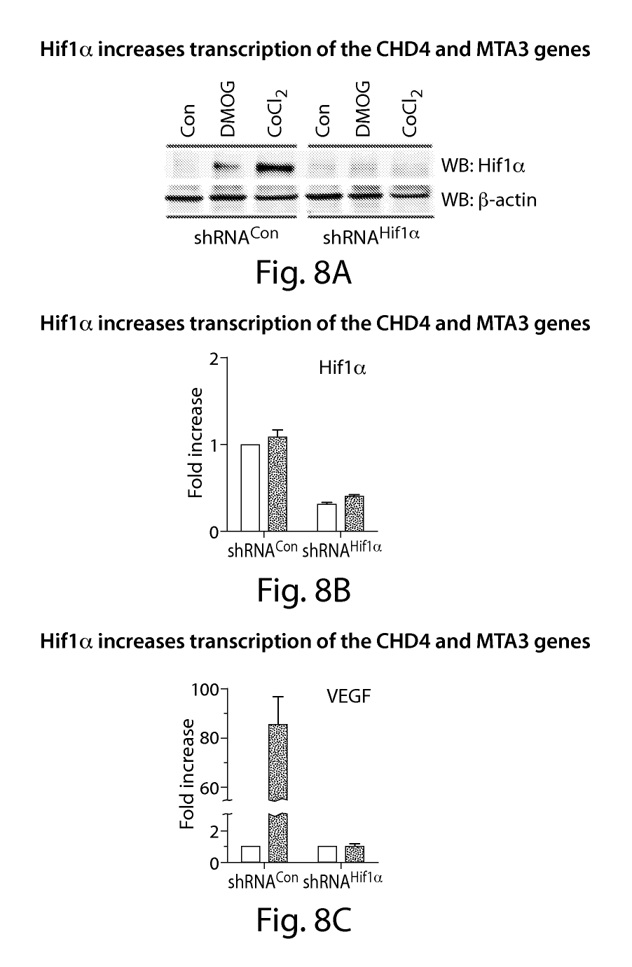

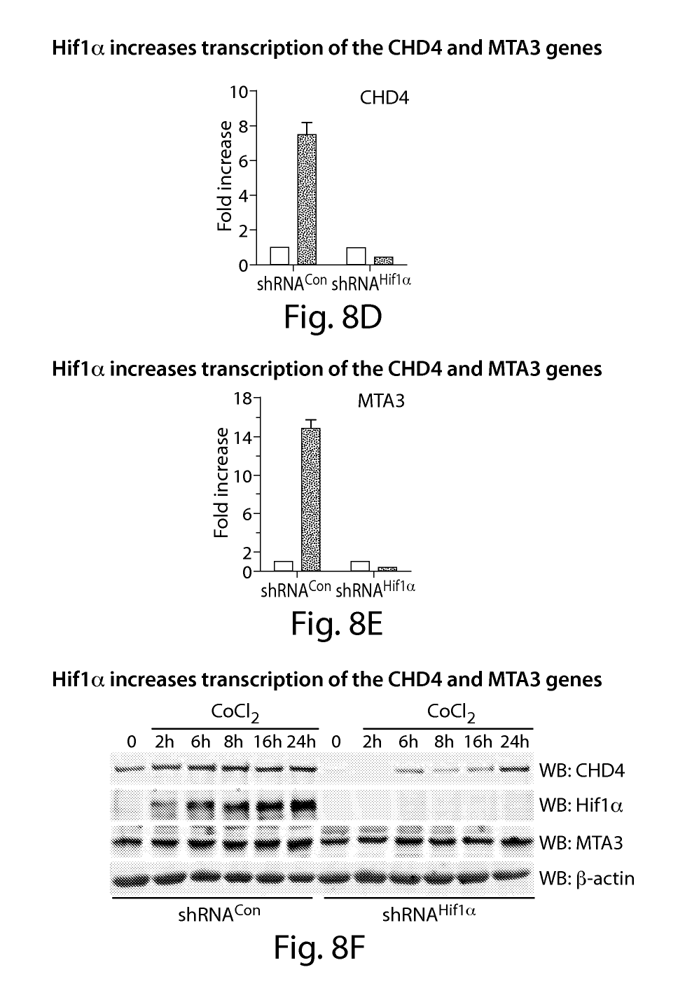

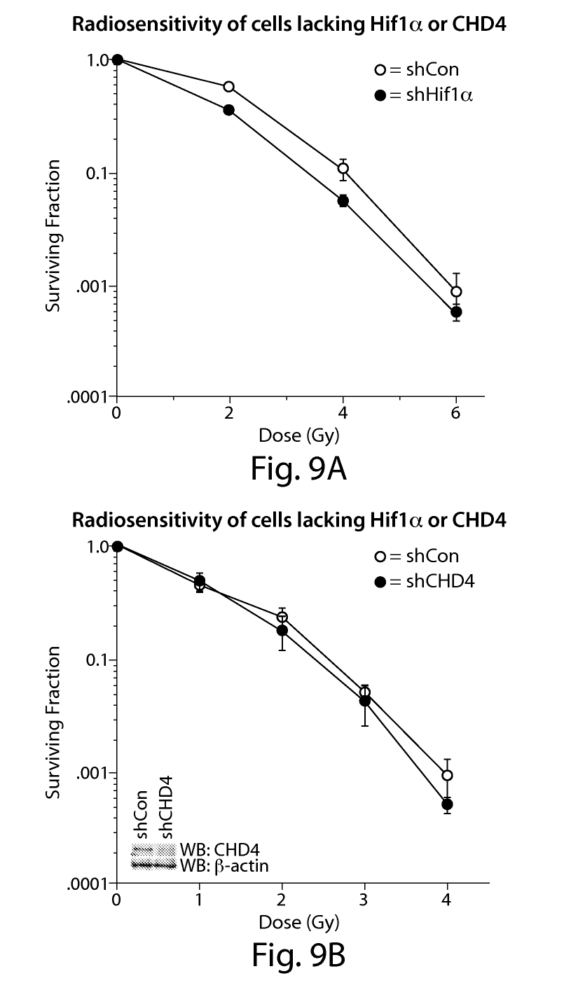

To examine the transcriptional activity of Hif1α, the inventors first reduced expression of Hif1α using shRNA. shRNA targeting Hif1α blocked the accumulation of Hif1α after treatment with either DMOG or CoCl₂ (FIG. 8A) and reduced the levels of Hif1α mRNA (FIG. 8B). Silencing of Hif1α also inhibited the accumulation of VEGF mRNA (FIG. 8C), a key target of Hif1α, demonstrating that Hif1α function is abolished in the shRNA^{HIF1α} cells. Next, we determined if Hif1α regulates expression of the CHD4 and MTA3 genes. FIG. 8D and 8E demonstrate that activation of Hif1α by CoCl₂ increased the levels of CHD4 and MTA3 mRNA, and that this was abolished when Hif1α was silenced with shRNA (FIGS. 8D and 8E). This demonstrates that Hif1α is required for the accumulation of CHD4 and MTA3 mRNA after exposure to CoCl₂. Further, CoCl₂ also increased the levels of Hif1α, CHD4 and MTA3 protein with similar kinetics (FIG. 8F), consistent with the Hif1α-dependent increase in their mRNA levels (FIGS. 8D and 8E). Significantly, suppression of Hif1α with shRNA also reduced the basal levels of CHD4 protein, whereas basal levels of MTA3 were unaffected by loss of Hif1α (FIG. 8E, 8F). Further, loss of Hif1α greatly attenuated the accumulation of CHD4 after exposure to CoCl₂, but had only a small impact on the accumulation of MTA3 protein (FIG. 8F). FIG. 8 therefore demonstrates that increased levels of Hif1α lead to increased levels of MTA3 and CHD4 mRNA, potentially identifying CHD4 and MTA3 as transcriptional targets for Hif1α. Significantly, loss of Hif1α decreased both the basal and stimulated levels of CHD4 protein, indicating that Hif1α plays a critical role in maintaining basal and stimulated levels of CHD4. Previous work indicates that CHD4 can participate in the cells response to IR-induced DNA damage. Cells expressing shRNA to Hif1α have decreased levels of CHD4, and should be more sensitive to IR. FIG. 9A demonstrates that cells lacking Hif1α exhibit a small but significant increase in radiosensitivity, consistent with a key role for Hif1α in regulating radiosensitivity. However, when shRNA was used to deplete CHD4 protein levels to a level similar to those detected in Hif1α depleted cells (compare FIGS. 8F and 9B, inset) no significant impact on radiosensitivity was seen (FIG. 9B). Similarly, silencing MTA3 expression with shRNA did not alter cellular radiosensitivity (data not shown). Thus, while Hif1α contributes to cell survival after exposure to IR (FIG. 7C and FIG. 9A), this regulation of radiosensitivity is not mediated through the ability of Hif1α to regulate the expression of either CHD4 or MTA3. Therefore, although CHD4 and MTA3 have been identified as potential transcriptional targets for Hif1α, the ability of Hif1α to protect cells from radiation damage does not require either CHD4 or MTA3. It is more likely that upregulating CHD4 and MTA3, which are components of NuRD deacetylase complex, plays a key role in other processes, such as transcriptional repression, which are a feature of the hypoxia response.

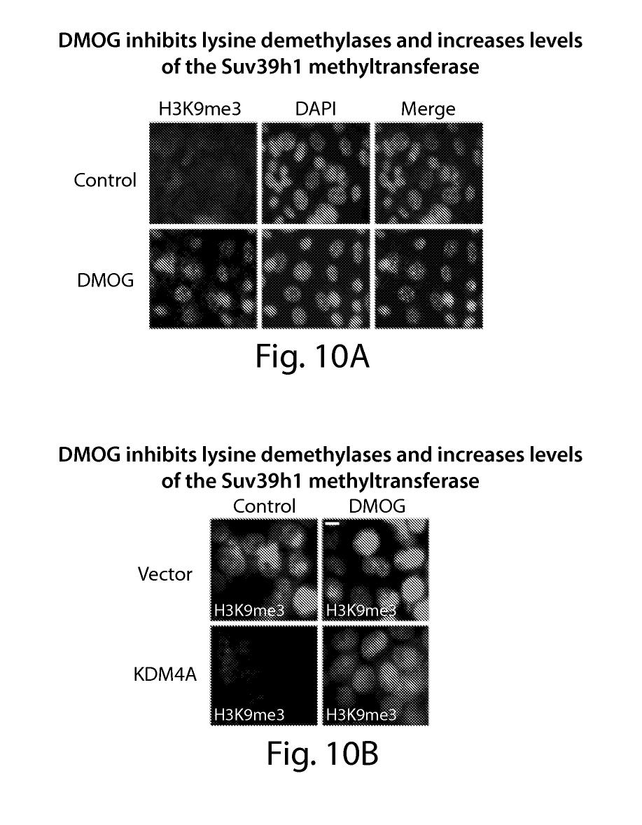

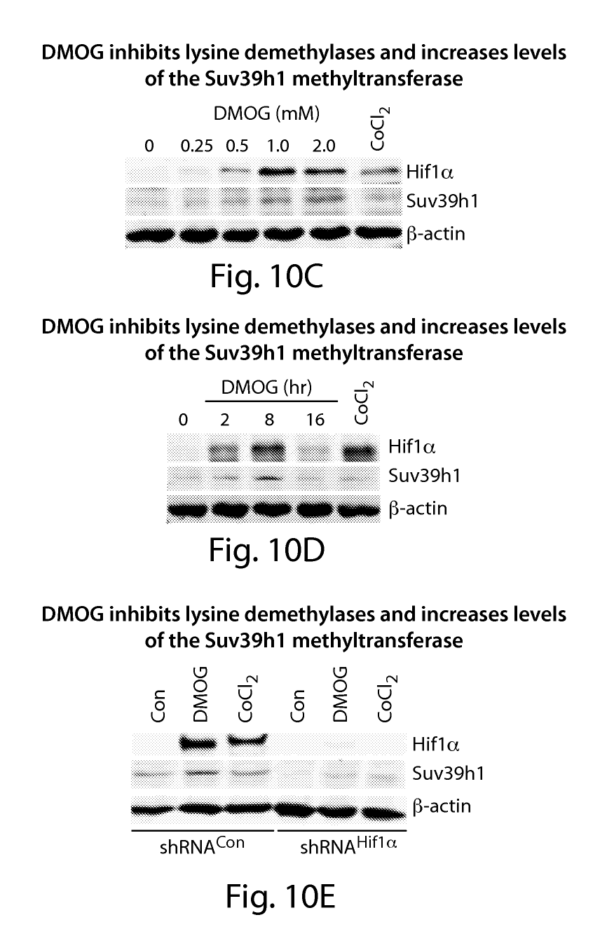

To further explore how stabilization of Hif1α can regulate radiosensitivity, a second group of proteins, the histone demethylases, which are transcriptionally activated by Hif1α were examined. Histone methylation is a dynamic signal transduction process, controlled by histone methyltransferases and histone demethylases (KDMs). Histone methylation plays a key role in regulating chromatin structure, transcriptional activity and in the DNA damage response. The importance of histone methylation in the DNA damage response is highlighted by the observation that inactivation of H3K9me3 methyltransferases leads to genomic instability and an inability to correctly repair DSBs caused by IR. Several lysine demethylases (KDMs), including KDM4B and KDM3A, contain Hypoxia Response Elements (HRE) and are transcriptionally activated by Hif1α under hypoxic conditions, suggesting that histone methylation may decrease under hypoxic conditions. Accordingly, how the stabilization of Hif1α by DMOG impacts methylation of H3K9me3 was examined. In FIG. 10A, exposure of cells to DMOG rapidly increased H3K9me3 in cells. This was somewhat unexpected, since Hif1α transcriptionally upregulates 2 H3K9me3 specific KDMs, KDM4B and KDM3A, and should therefore decrease H3K9me3 levels. However, KDMs and the Hif1α prolylhydroxylases belong to the larger family of dioxygenases. Both the Hif1α prolylhydroxylases and KDMs utilize an Fe (II) and 2-oxoglutarate-dependent dioxygenase mechanism to either hydroxylate proline on Hif1α or remove methyl groups from methylated lysines on histone tails. DMOG, which is a 2-oxoglutarate analog, is a competitive inhibitor of the Hif1α-PH and is therefore likely to inhibit KDMs as well. In FIG. 10B, the H3K9me3 specific demethylase KDM4A was transiently expressed in cells. Cells expressing vector showed increased H3K9me3 methylation when cells were exposed to DMOG, consistent with inhibition of endogenous KDMs. Overexpression of KDM4A resulted in almost complete loss of endogenous H3K9me3 in the cells; however, addition of DMOG to cells expressing KDM4A restored H3K9me3 levels to near normal. FIG. 10B therefore clearly demonstrates that DMOG, in addition to increasing Hif1α protein levels, can also inhibit endogenous KDMs and therefore increase levels of H3K9me3 in the cells.

To further analyze how DMOG may regulate H3K9me3 levels, it was also determined if Hif1α could increase expression of Suv39h1, a key H3K9me3 methyltransferase. Surprisingly, FIGS. 10C and 10D demonstrate that both DMOG and, to a lesser extent, CoCl₂, increased expression of the Suv39h1 methyltransferase. Further, the increase in Suv39h1 protein closely followed the dose-dependent (FIG. 10C) and time-dependent (FIG. 10D) increase in Hif1α levels caused by DMOG. Importantly, both basal and DMOG dependent induction of Suv39h1 were abolished in cells lacking Hif1α (FIG. 10E), indicating that Hif1α directly regulates the levels of Suv39h1 in cells. FIG. 10 therefore demonstrates that DMOG can influence H3K9me3 levels by directly inactivating H3K9 demethylases (FIGS. 10A and 10B) as well as upregulating H3K9 methyltransferases through a Hif1α dependent mechanism (FIG. 10C-E).

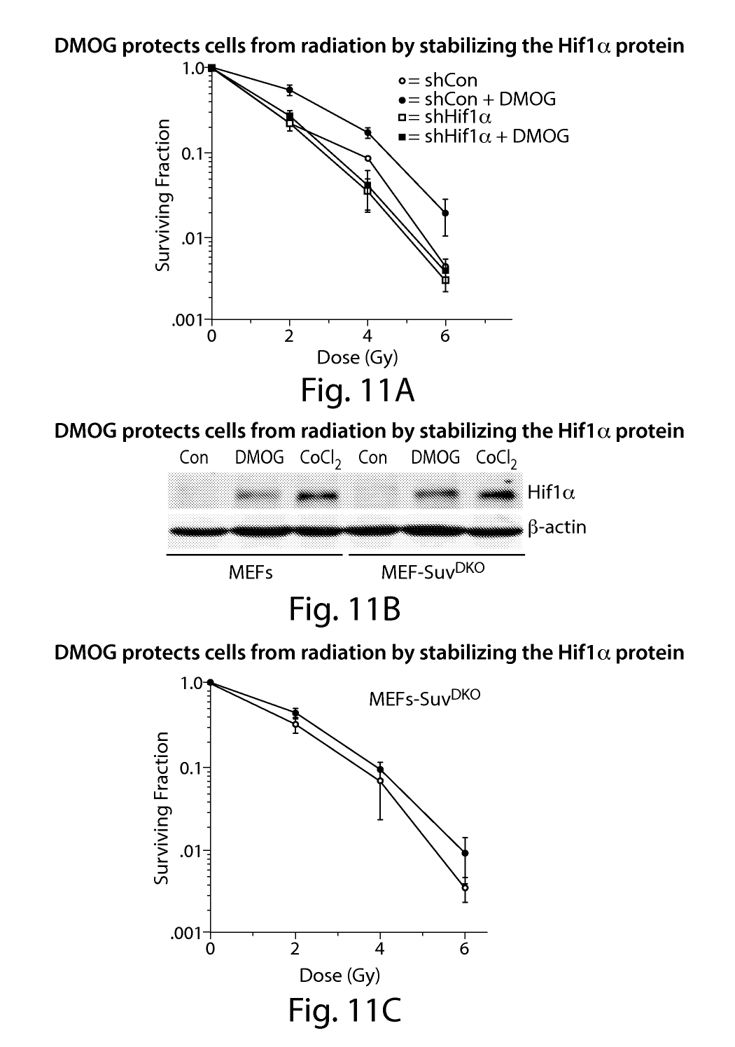

The previous results indicate that DMOG may influence radiosensitivity through 2 distinct pathways. First, DMOG can directly inhibit KDMs, increasing H3K9me3 levels by a mechanism which is independent of Hif1α. Second, DMOG can directly inhibit Hif1α-prolylhydroxylases, leading to stabilization and accumulation of transcriptionally active Hif1α. To determine the relative contributions of these 2 pathways to the ability of DMOG to function as a radioprotector, the inventors examined how silencing of Hif1α with shRNA impacted radioprotection. FIG. 11A demonstrates that DMOG increased radioresistance in MEFs expressing a non-specific shRNA, but this effect was lost when Hif1α was silenced. The ability of DMOG to function as a radioprotector therefore requires Hif1α, indicating that DMOG exerts its primary effect on radiosensitivity through inhibition of the Hif1α -prolylhydroxylase and accumulation of Hif1α. Therefore, although DMOG can also inhibit KDMs (FIGS. 10A and 10B), this inhibition does not appear to be critical for the observed radioprotection.

Finally, we examined if the Hif1α-dependent increase in expression of the Suv39h1 methyltransferase was required for DMOG dependent radioprotection. For this, we determined if DMOG can protect MEFs lacking both the Suv39h1 and Suv39h2 methyltransferases (Suv^{DKO}cells). The ability of DMOG and CoCl₂ to increase Hif1α levels was not altered in the Suv^{DKO} MEFs (FIG. 11B). Further, whereas DMOG functioned as a radioprotector in normal MEFs (FIG. 11A), the protective effect of DMOG was significantly reduced in the Suv^{DKO} MEFs (FIG. 11C). This indicates that the reduced ability of DMOG to protect SuvDKO MEFs may be attributed to both the loss of Hif1α dependent increase in the Suv39h1 methyltransferase, as well as changes in other Hif1α target genes. Overall, FIG. 11 demonstrates that DMOG can protect cells form radiation by stabilizing the Hif1α transcription factor, and increasing expression of genes, including the Suv39h1 methyltransferase, which can promote cell survival

Finally, it was determined if DMOG could protect cells from DNA damage at the level of the whole organism. Hif1α activation can transcriptionally activate a wide range of genes, including growth factors, such as VEGF, which promotes angiogenesis, and erythropoietin production, which mobilizes bone marrow and improves blood parameters. It is likely that these factors may be important in promoting recovery of sensitive tissues, such as the bone marrow and gastrointestinal tract, from total body irradiation. In order to assess if the radioprotective effects of DMOG can be detected at the level of the whole organism, it was examined if DMOG had protective effects in a murine total body irradiation (TBI) model. As shown in FIG. 6 and discussed in Example 2, activation of Hif1α by DMOG results in the transcriptional upregulation of many genes and growth factors and is associated with increased cell survival in culture and protection of mice form whole body irradiation. These results clearly demonstrated that DMOG exhibits radioprotective potential in murine models.

The Hif1α transcriptional regulatory protein controls the expression of genes which promote cell survival under conditions of low oxygen tension. if1α can switch cell metabolism towards increased expression of growth factors and anti-apoptotic factors as well as switching of metabolic pathways allow the cell to maintain energy levels under hypoxic conditions. Hif1α levels are controlled through direct hydroxylation of Hif1α by the PHD2 prolylhydroxylase, leading to degradation of Hif1α under normal oxygen tension. Small molecule inhibitors of prolylhydroxylases have been developed which inhibit PHD2 and related enzymes, leading to stabilization of Hif1α and upregulation of the hypoxia response. Here, we have shown that stabilization of Hif1α using the prolylhydroxylase inhibitor DMOG protects both MEFs and mice from the cytotoxic effects of exposure to IR. This is consistent with previous studies demonstrating that tumor cells containing constitutively high levels of Hif1α are more resistant to both chemotherapy and radiotherapy. Increasing Hif1α levels in normal cells with prolylhydroxylase inhibitors such as DMOG therefore represents a novel pathway for the development of new and effective radioprotective agents.

Our results clearly show that DMOG requires Hif1α to protect cells from radiation, since suppression of Hif1α with shRNA abolished the protective effect of DMOG. A key question is to determine how stabilization of Hif1α by DMOG can promote radioprotection. Because Hif1α functions as a transcriptional activator, it likely exerts its protective effects through increased expression of genes involved in DNA repair or cell survival. Previous studies indicate that hypoxia can alter expression of components of the mismatch repair pathway; however, altered expression of these proteins would not account for radioprotection observed after activation of Hif1α by DMOG. Nevertheless, 3 new targets for Hif1α have been identified—the CHD4 helicase, the MTA3 regulatory protein and the Suv39h1 methyltransferase. All 3 were increased at both the mRNA and protein level by DMOG and the hypoxia mimetic agent CoCl₂ Further, this increase in mRNA required the Hif1α transcription factor, suggesting that Hif1α can directly regulate their transcription. Previous work has shown that MTA1, a close family member related to MTA3, is also transcriptionally upregulated by Hif1α during hypoxia, implying that the MTA family of coactivators are a common target for the Hif1α transcription factor. Both CHD4 and MTA3 are components of the NuRD complex, a histone deacetylase complex which is implicated in DNA repair. Inactivation of CHD4 leads to increased sensitivity to IR-induced DNA damage, indicating a key role in the repair of DSBs. Taken together, this would suggest that increased levels of NuRD may protect cells from radiation. However, although Hif1α was important for DMOG to protect cells from IR, loss of either CHD4 or MTA3 expression did not alter sensitivity to IR in the cell system. Thus, although DMOG can stabilize Hif1α and increase levels of the NuRD complex, the accumulation of NuRD does not significantly impact the radiosensitivity of the cells. The accumulation of the NuRD deacetylase complex may therefore play an alternate role in the hypoxia response, such as transcriptional repression of genes during low oxygen tension.

An alternate explanation for how DMOG may protect cells from radiation damage can be proposed based on the previous observation that Hif1α can alter expression of genes involved in the regulation of histone methylation. Hif1α increases expression of histone demethylases, including KDM4A and KDM4B, which function to remove methyl groups from methylated histones on the chromatin. The increased expression of KDMs during hypoxia is associated with a decrease in histone methylation, including a reduction on H3K9me3 levels. Previous work has shown that a decrease in H3K9me3 levels is associated with an increase in radiosensitivity. Hif1α-dependent increases in KDM expression would therefore be predicted to increase, rather than decrease, radiosensitivity. However, it was found that, unlike hypoxia, DMOG increased rather than decreased H3K9me3 levels. This result is explained by the fact that KDMs and Hif1α-prolylhydroxylases share a common catalytic mechanism, so that both classes of enzyme are inhibited by DMOG (FIG. 10B). DMOG therefore inhibits the Hif1α-prolylhydroxylase, leading to accumulation of transcriptionally active Hif1α and upregulation of KDMs. However, because DMOG can also directly inhibit KDMs (FIG. 10B), this essentially negates the increased expression of KDMs mediated by Hif1α. Overall, DMOG will increase histone methylation through inhibition of KDMs. Further, cells in which Hif1α was inactivated were not protected by DMOG, despite the ability of DMOG to increase H3K9me3 levels in these cells. Thus, the ability of DMOG to protect cells from IR is mediated through the transcriptional activity of Hif1α rather than through a general inhibition of endogenous KDMs in the cell.

In addition, to CHD4 and MTA3, the Suv39h1 methyltransferase was also identified as a target of the Hif1α. Suv39h1 is a key player in the di- and trimethylation of H3 on lysine 9. Further, loss of Suv39h1 and decreased levels of H3K9me3 are associated with increased radiosensitivity and decreased genomic stability. Secondly, DMOG can also increase the expression of Suv39h1 through activation of Hif1α, a process which will also tend to increase H3K9me3 levels. Importantly, the ability of DMOG to function as a radioprotector was significantly reduced (but not abolished) in MEFs which lacked expression of Suv39h1 and Suv39h2. Since DMOG increases Suv39h1 expression through a Hif1α dependent mechanism, this indicates that the main contributor to DMOG mediated radioresistance is the transcriptional upregulation of the Suv39h1 methyltransferase by Hif1α. How does increased expression of Suv39h1 impact radiosensitivity? Cells lacking Suv39h1 have significant defects in both H3K9me3 and in DSB repair. DMOG may therefore increase expression of Suv39h1, leading to increased methylation of H3K9 and allowing for more efficient activation of the DNA damage response. Suv39h1 may therefore methylate H3K9 within specific regions of the chromatin after DNA damage in order to improve the efficiency of repair. However, it is also possible that the radioprotective effects of increased Suv39h1 are not directly on the DNA repair machinery, but instead feedback through altered methylation of key genes, such as anti-apoptotic proteins. Overall, the protective effect of DMOG is largely mediated through the Hif1α dependent increase in expression of the Suv39h1 methyltransferase, leading to increased H3K9me3 levels in the cell.

Finally, it is also demonstrated that when DMOG is given to mice prior to irradiation it can protect them from total body irradiation. Significant improvement in survival was found in 2 different mouse strains, underlining the effectiveness of DMOG in a whole animal model. Previous studies using DMOG and related prolylhydroxylase inhibitors in whole animal models have indicated protection from ischemic injury, protection in a murine model of colitis and the development of hypoxia tolerance. A key target of Hif1α are the growth factors VEGF and erythropoietin. DMOG can detectably increase erythropoietin levels in animal models, leading to improvement in blood parameters, and can act to increase angiogenesis and muscle recovery from ischemic injury. The most sensitive tissues to IR are the GI tract and bone marrow. The ability of DMOG to stimulate the production of factors such as erythropoietin and VEGF, which can stimulate repopulation of the hematopoietic progenitors and promote formation of new vasculature, are likely to be critical factors in the ability of DMOG to protect whole animals form radiation.

Effective radioprotectors and radiation-mitigating agents are needed in the clinic to treat the radiation victims and to protect individuals from radiation exposure resulting from nuclear disasters or radiological attack. Many small molecules, including anti-oxidants, cytokines, activators of NF-KappaB and cyclin-dependent kinase inhibitors have been shown to have radioprotective effects in murine TBI models. These results demonstrate that the prolyl hydroxylase inhibitor DMOG is an effective radioprotector in both tissue culture and whole animal models. Activation of Hif1α evokes a complex response at both the cellular and whole organism levels. Changes in gene transcription caused by DMOG at the cellular level, including changes in histone methylation, can impact the ability of individual cells to repair and survive radiation exposure. In addition, the ability of DMOG to stabilize Hif1α and stimulate production of growth factors such as VEGF and erythropoietin can promote DNA repair, the repopulation of sensitive cell types and promote survival at both the level of individual tissues and the whole organism. Manipulation of the Hif1α transcriptional pathway may reveal new targets for the development of novel radioprotective agents.

This invention is not limited in its application to the details of construction and the arrangement of components set forth in the above description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

We claim:

1. A method to inhibit tissue injury in a subject resulting from exposure to radiation, the method comprising:
administering to the subject in need thereof a prolyl hydroxylase (PH) inhibitor in an amount effective to inhibit tissue injury caused by radiation exposure of the subject, wherein the tissue is non-cancerous.

2. The method of claim 1, wherein the radiation exposure results from radiation therapy.

3. The method of claim 1, wherein the inhibitor is administered between 1 day before and 3 days after exposure of the subject to the radiation exposure.

4. The method of claim 1, wherein the inhibitor is administered after the radiation exposure, but within 48 hours of the radiation exposure.

5. The method of claim 1, wherein the inhibitor is a non-specific PH inhibitor.

6. The method of claim 1, wherein the inhibitor is an inhibitor of HIF1α-prolyl hydroxylase or of histone lysine demethylase (KDM).

7. The method of claim 1, wherein the inhibitor is selected from the group consisting of: dimethyloxalylglycine (DMOG), N-oxalylglycine (NOG), desferrioxamine (DFO), FG-4383, F-0041, FG-2216, FG-4592, S956711, ethyl-3,4 dihydroxy-benzoate (EDHB), TM6089, TM655, TM6008 and 8-hydroxyquinoline derivatives.

8. The method of claim 1, wherein the inhibitor is administered parenterally.

9. The method of claim 1, wherein the inhibitor is administered orally.

10. A method comprising:
contacting non-cancerous cells with a prolyl hydroxylase (PH) inhibitor in an amount effective to reduce the number of double-stranded DNA breaks (DSB) in the cells, wherein the cells have been, are or will be exposed to radiation causing double stranded DNA breaks.

11. The method of claim 10, wherein the cells are contacted with the inhibitor between 1 day before and 3 days after exposure of the cells to the radiation.

12. The method of claim 10, wherein the cells are contacted with the inhibitor after exposure of the cells to the radiation, but within 48 hours of exposure of the cells to the radiation.

13. The method of claim 10, wherein the inhibitor reduces the number of DSB in the cell by at least 5%, 10%, 20%, 30%, 40% or 50% as compared to the number of DSB in control cells not exposed to conditions causing DSB.

14. The method of claim 10, wherein the inhibitor is a non-specific PH inhibitor.

15. The method of claim 10, wherein the inhibitor is an inhibitor of HIF1α-prolyl hydroxylase or of histone lysine demethylase (KDM).

16. The method of claim 10, wherein the inhibitor is selected from the group consisting of: dimethyloxalylglycine (DMOG), N-oxalylglycine (NOG), desferrioxamine (DFO), FG-4383, F-0041, FG-2216, FG-4592, S956711, ethyl-3,4 dihydroxy-benzoate (EDHB), TM6089, TM655, TM6008 and 8-hydroxyquinoline derivatives.
